(19) United States
(12) Patent Application Publication
(10) Pub. No.: US 2026/0234540 A1
(43) Pub. Date: Aug. 13, 2026

(54) INDIRECT SONICATION FOR CELL EXTRACT PREPARATIONS FOR CELL-FREE PROTEIN SYNTHESIS

(71) Applicant: National Resilience, Inc., San Diego, CA (US)

(72) Inventors: Danielle Yoesep, San Diego, CA (US); Krishnathreya Satish, San Diego, CA (US); Weston Kightlinger, San Diego, CA (US)

(21) Appl. No.: 19/156,776

(22) PCT Filed: Feb. 16, 2024
(86) PCT No.: PCT/US2024/016278

§ 371 Date: Aug. 14, 2025

Related U.S. Application Data
(60) Provisional application No. 63/446,781, filed on Feb. 17, 2023.

(51) Int. Cl. C12N 1/066; C12M 1/00; C12N 1/165; C12N 1/205; C12N 5/04; C12N 5/07; C12N 5/071; C12N 13/00; C12P 21/00

(52) U.S. Cl. CPC C12N 1/066; C12M 47/06; C12N 1/165; C12N 1/205; C12N 5/04; C12N 5/0601; C12N 5/0602; C12N 13/00; C12P 21/00; C12N 2521/10

(57) ABSTRACT

Methods of lysing cells, e.g., for use in methods of cell-free protein synthesis, are disclosed that include placing one or more vessels within a liquid in a reservoir, wherein each vessel contains one or more cells in a cell suspension; and applying sonic energy to the liquid within the reservoir during a plurality of predetermined time intervals and under conditions sufficient to disrupt cell walls and/or cell membranes of cells in the cell suspension, and to release into the cell suspension cell contents that include active transcription and/or translation machinery.

### CLAIM OF PRIORITY

This application claims the benefit of U.S. Provisional Application Ser. No. 63/446,781, filed on Feb. 17, 2023. The entire contents of the foregoing are incorporated herein by reference.

### TECHNICAL FIELD

The present disclosure relates generally to devices, systems, and methods for lysing cells with a high throughput to generate active cell extracts for use in cell-free protein synthesis.

### BACKGROUND

Cell extracts contain transcription and translation machinery that is required for cell-free protein synthesis (CFPS). Disrupting cell membranes, e.g., through cell lysis, releases the cell extracts from the cells so that cell-free protein synthesis can occur. However, prior attempts at cell lysis have also caused the destruction of the cell machinery required for CFPS.

### SUMMARY

The present disclosure provides devices, systems, and methods for lysing cells efficiently and with a low coefficient of variation for use in cell-free protein synthesis experimentation and is based, at least in part, on the discovery that by using indirect sonication with careful control of certain sonication parameters one can obtain cell extracts at a high throughput in small volumes and with highly active transcription and translation machinery. This high-throughput cell lysis improves the process of generating cell extracts and provides a low coefficient of variation between samples of extracts from the same cellular material, which is desirable to more readily attribute differences between samples to the samples themselves rather than to the lysis method.

In general, the cells are in a cell suspension, optionally divided between a plurality of vessels, e.g., a sample tray of 96 vessels. The vessels, e.g., in a sample tray, are placed in a liquid-filled reservoir and sonic energy is applied to the liquid in the reservoir to disrupt the cell walls and/or cell membranes and cause the cells to release a cell extract. A transducer or sonic horn provides the sonic energy at appropriate amplitudes and time intervals to disrupt the cell wall and/or cell membrane without destroying the transcription and translation machinery contained within the cell, e.g., so that the transcription and translation machinery can later be used to synthesize proteins with a high level of activity after release from the cells. In other embodiments, the cell suspension is within the lumen of a pipe or other continuous flow device surrounded by the liquid to which the sonic energy is applied.

In one aspect, the disclosure provides methods of lysing cells within vessels, e.g., in a sample plate or tray, within a liquid in a reservoir, wherein each vessel contains one or more cells, e.g., bacterial cells such as E. coli (e.g., in S30 buffer), yeast cells, rabbit reticulocytes, wheat germ, other mammalian cells such as Chinese hamster ovary (CHO) cells, or Sf21 worm ovary cells, in a cell suspension. The methods include applying sonic energy, e.g., ultrasonic energy, to the liquid within the reservoir in predetermined time intervals and under conditions sufficient to disrupt cell walls and/or cell membranes and to release into the cell suspension cell contents including active transcription and translation machinery, e.g., capable of synthesizing protein from DNA templates, in other words, the sonic energy is applied under conditions sufficient to maintain the functionality of the transcription and translation machinery contained within the cells.

In another aspect, the disclosure provides methods of cell-free protein synthesis that include placing a plurality of vessels, e.g., in a sample plate or tray, within a liquid-filled reservoir, wherein each vessel contains one or more cells, e.g., bacterial cells such as E. coli (e.g., in S30 buffer), yeast cells, rabbit reticulocytes, wheat germ, other mammalian cells such as Chinese hamster ovary (CHO) cells, or Sf21 worm ovary cells, in a cell suspension, applying sonic energy, e.g., ultrasonic energy, to the liquid within the reservoir in predetermined time intervals and under conditions sufficient to disrupt cell walls and/or cell membranes and to release into the cell suspension cell contents that include active transcription and/or translation machinery, harvesting and/or processing at least a portion of the cell contents, and synthesizing proteins using the transcription and/or translation machinery within the cell content.

In general, the transcription and/or translation machinery includes one or more or all of ribosomes, tRNAs, synthetases, transcription factors, translation initiation factors, elongation factors, and release factors.

Embodiments of these methods can include one or any combination of two or more of the following features, embodiments, and implementations.

In various embodiments, the sonic energy is applied indirectly to the cell suspension during a total time period of about 30 seconds to about 6 hours, e.g., about 30, 40, 50, 60, 70, 80, 90, 100, or 120 seconds, or about 1, 2, 3, 4, 5, 6, 7, 8, 9, 10, 15, 20, 25, 30, 40, 50, or 60 minutes, or about 1, 2, 3, 4, 5, or 6 hours.

In certain embodiments, the sonic energy is applied to the liquid and indirectly to the cell suspension during the total time period by applying the sonic energy during a plurality of predetermined time intervals of about 5 to about 120 seconds each, e.g., about 5, 10, 15, 20, 25, 30, 35, 40, 45, 50, 55, 60, 70, 80, 90, 100, 110, or 120 seconds, separated by gaps of about 5 to about 30 seconds, e.g., about 5, 10, 15, 20, 25, or 30 seconds, during which sonic energy is not applied, or the sonic energy is reduced.

In some embodiments, the predetermined time intervals are each about 5 seconds to about 20 seconds and the gaps between predetermined time intervals is about 10 to about 20 seconds.

In some embodiments, the total time during which the sonic energy is applied is greater than or equal to about 10 minutes, up to about 1 hour, e.g., up to about 15, 20, 25, 30, 40, 50, or 60 minutes.

In certain embodiments, the sonic energy is equal to an energy delivered by a sonicator with an amplitude greater than 50%, or the amplitude of the sonic energy is equal to an amplitude delivered by a sonicator with an amplitude set at greater than 50%, or a power of the sonic energy is equal to a power delivered by a sonicator with an amplitude set at greater than 50%.

In some embodiments, a power density (W/mL reservoir) of the sonic energy is equal to the power density delivered by a sonicator with an amplitude greater than 50%, or an energy density (J/mL reservoir) of the sonic energy is equal to an energy density delivered by a sonicator with an amplitude set at greater than 50%, or a power density of the sonic energy is within a range of about 2 to about 3 W/mL of a volume of the reservoir.

In certain embodiments, an energy density of the sonic energy is within a range of about 2000 to about 5000 J/mL of a volume of the reservoir.

In some embodiments, the methods include maintaining a temperature of the liquid within the reservoir below a threshold temperature, e.g., to avoid overheating and destroying or reducing the functionality of the transcription and translation machinery.

In various embodiments, the threshold temperature is at or below about 50° C., 40° C., 35° C., 30° C., 25° C., 20° C., 15° C., 10° C., or 5° C.

In some embodiments, the temperature of the liquid within the reservoir is maintained with a cooling system.

In some embodiments, the temperature of the liquid within the reservoir is maintained by controlling a length of the predetermined time interval and/or a length of the gap between the predetermined time intervals.

In various embodiments, the methods include transmitting the sonic energy through a sonic horn, and the vessels are part of a sample tray or plate, e.g., a multi-well plate, such as a multi-well plate that includes 6, 24, 48, 96, 384, or 1536 wells.

In some embodiments, the sample tray or plate includes a Society for Biomolecular Screening (SBS) standardized plate.

In various embodiments, the cells include bacterial cells, yeast cells, mammalian cells, insect cells, plant cells, and/or worm cells.

In general, the frequency of the sonic energy is in a range of about 15 and about 30 kHz, e.g., 15, 16, 7, 18, 19, 20, 21, 22, 23, 24, 25, 26, 27, 28, 29, or 30 kHz.

In general, the volumes of the cell suspensions are in a range between about 20 microliters and about 50 milliliters, e.g., 20, 30, 40, 50, 60, 70, 80, 90, 100, 250, 500, 750, or 1000 microliters, or about 1, 5, 10, 15, 20, 25, 30, 35, 40, 45, or 50 milliliters, and the liquid within the reservoir can be one or more of water, one or more buffers, one or more gels, and/or one or more organic solvents.

In some embodiments, the vessels each have a volume of less than 2 milliliters.

In some embodiments, cooling the reservoir and/or intervals of applying sonic energy are used to maintain a temperature within the reservoir, e.g., of the cell suspension in the reservoir, below a temperature threshold, e.g., a temperature at or below about 50° C., 40° C., 35° C., 30° C., 25° C., 20° C., 15° C., 10° C., or 5° C.

In general, synthesizing the proteins includes combining the portion of the cell contents with one or more nucleic acid templates that encodes for one or more proteins.

The devices, systems, and methods disclosed herein are advantageous in that the number of samples, e.g., in a sample tray, increases efficiency and throughput in cell lysis for experimental purposes. Other advantages include providing samples with low volumes, making the screening process for viable cell extracts much more efficient. Additional advantages include enabling a touchless lysis system, which can prevent loss of sample and cross-contamination. Therefore, the present devices, systems, and methods increase the yield and quality of cell lysis for experimenting with cell extracts for use in cell-free protein synthesis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

Other features and advantages of the invention will be apparent from the following detailed description, and from the claims.

Sonic waves, as used herein, include infrasonic waves, e.g., below 16 Hz, sonic waves, e.g., between 16 Hz and 20 Hz, and ultrasonic waves, e.g., above 20 Hz. The application of sonic energy, e.g., sonication, can include applying infrasonic waves, sonic waves, and/or ultrasonic waves.

### DESCRIPTION OF DRAWINGS

FIG. 1 is a schematic diagram of one example of a system for cell lysis.

FIG. 2 is another schematic diagram of the system of FIG. 1.

FIG. 3 is a schematic diagram that compares the efficiencies of different modes of direct sonication and indirect sonication.

FIG. 4 is a bar graph illustrating the average cell-free superfolder green fluorescent protein (sfGFP) reporter production enabled by direct sonication and indirect sonication.

FIG. 5 is a bar graph illustrating the average cell-free sfGFP reporter production enabled by direct sonication and indirect sonication.

### DETAILED DESCRIPTION

The present disclosure provides devices, systems, and methods for lysing cells efficiently and with a low coefficient of variation. The cells are contained in a cell suspension divided between a plurality of vessels, e.g., within a sample tray of vessels, e.g., a sample tray or plate of 6, 24, 48, 96, 384, or 1536 wells. The sample tray is placed in a liquid-filled, e.g., water-filled, reservoir, and sonic energy is applied to the liquid to disrupt the walls and/or membranes of the cells within the vessels indirectly and to release cell extracts from the cells without impairing the transcription and translation machinery. The transcription and translation machinery can then be used to synthesize proteins, e.g., via cell-free protein synthesis.

### Methods of High-Throughput Cell Lysis

Cells contain transcription and translation machinery (e.g., enzymes, helper factors, catalysts, ribosomes, etc.) that can synthesize and fold desired protein products. A user can extract the transcription and translation machinery, e.g., in the form of cell extract, to synthesize and fold the desired protein products outside of the cell, i.e., using cell-free protein synthesis. First, the cells can be cultured, e.g., within shake flasks, test tubes, etc., to grow the cells and produce the transcription and translation machinery. If the cell pellets produced from these cell cultures are then frozen for storage, they must first be thawed for use in the present methods. The user can then suspend the cells in a liquid, e.g., a buffer (e.g., acetate salt buffer) and then distribute the cell suspension across a plurality of vessels, e.g., in a sample plate or tray. For example, the user can distribute the cell suspension equally across the vessels. In some implementations, the user can store the sample plate, e.g., on ice, in a refrigerator, etc., for a period of time before performing cell lysis.

A system, e.g., as described herein, is used to perform cell lysis using the methods described herein. The systems can include a liquid-filled, e.g., water-filled, reservoir, and includes a source of sonic energy to apply the sonic energy to the liquid to indirectly disrupt the walls and/or membranes of the cells within the vessels and release cell extracts without impairing the transcription and translation machinery. Once the system is prepared for use, the user can place the sample plate containing the cellular suspensions into a plate holder within the liquid-filled reservoir and perform a sonication, e.g., an ultrasonication, protocol to sonicate the cells in the cell suspension indirectly. By using this indirect method, rather than direct sonication, the cells walls and/or membranes can be disrupted at a higher efficiency and throughput.

Once the sonication process is complete, the user removes the vessels, e.g., the sample plate, from the plate holder and carries out post-lysis processing of the cell extracts. For example, the user can centrifuge the prepared extracts to clarify the extract, e.g., separating the cell debris from the translation and transcription machinery. In some implementations, the user transfers the samples from the sample plate into other vessels, e.g., individual microtubes or eight-strip microtubes, before centrifugation. Post-lysis processing can also include a run-off reaction, which includes supernatant incubation and agitation after the centrifugation. In some implementations, the user can centrifuge the prepared extracts a second time after the run-off reaction. By avoiding the transfer of cellular debris into the final extract, a user can produce extracts without remaining cells, e.g., for cell-free protein synthesis.

Cell lysis, as described herein, can be used to break down a variety of cells. For example, the methods described above can lyse bacterial cells (e.g., E. coli) and harvest their transcription and translation machinery. The methods described can also lyse mammalian cells (e.g., human cells, CHO cells, rabbit cells, and the like). Unlike mammalian cells, bacterial cells do not contain a nucleus and rarely harbor membrane-bound organelles. Bacterial cells can also often include cell walls, which mammalian cells do not have. The cell walls that surround the bacterial cells can be tough, rigid, and difficult to disrupt. Typically, bacterial cells can be much harder to lyse, e.g., without disrupting the transcription and translation machinery, than mammalian cells.

The methods described herein include a variety of amplitude ranges, power ranges, power density ranges, and frequency ranges to sonicate the cell suspensions and produce extracts indirectly without disrupting the transcription and translation machinery. Amplitude can be expressed in percentages, for example, where the amplitude percentage can reflect the voltage output of the signal generator. For example, a 100% amplitude may correspond to 1000 root-mean-square voltage (VRMS), which would represent the maximum power produced by the sonicator, a 50% amplitude may correspond to 500 VRMS, etc. For example, the QSonica Q700 can generate active cell extracts for cell-free protein synthesis at amplitude ranges between 50-100%. The QSonica Q700 device has a theoretical maximum power rating of 700 W (although in actual operation, the achievable maximum power rating may deviate from the theoretical power rating, e.g., 600-700 W) and an operating frequency of about 20 kHz.

In some implementations, the energy of the sonication is equal to an energy delivered by the sonication device with an amplitude greater than 50%. In some implementations, the energy of the sonication is less than an energy delivered by the sonication device with an amplitude greater than 50%. In some implementations, an amplitude of the sonic energy is equal to the amplitude delivered by the sonication device with an amplitude greater than 50%. In some implementations, an amplitude of the sonic energy is less than an amplitude delivered by the sonication device with an amplitude greater than 50%. In some implementations, a power of the sonication is equal to the power delivered by the sonication device with an amplitude greater than 50%. In some implementations, a power of the sonication is less than a power delivered by the sonication device with an amplitude greater than 50%. In some implementations, a power density (W/mL reservoir) of the sonication is equal to the power density delivered by the sonication device with an amplitude greater than 50%. In some implementations, the power density of the sonication is less than a power density delivered by the sonication device with an amplitude greater than 50%. In some implementations, an energy density (J/mL reservoir) of the sonication is equal to the energy density delivered by the sonication device with an amplitude greater than 50%. In some implementations, the energy density of the sonication is less than an energy density delivered by the sonication device with an amplitude greater than 50%.

In some implementations, the power density of the sonication is in a range of about 2 to about 3 W/mL of the reservoir volume. In some implementations, the energy density of the sonication is in a range of about 2000 to about 5000 J/mL of the reservoir volume, e.g., about 2000, about 2500, about 3000, about 3500, about 4000, about 4500, or about 5000 J/mL. In some implementations, the frequency of the sonic energy is in a range of about 15 to about 30 kHz, e.g., about 15, 16, 17, 18, 19, 20, 21, 22, 23, 24, 25, 26, 27, 28, 29, or 30 kHz.

The methods described herein include a variety of sonication times to indirectly sonicate the cell suspensions and produce extracts without disrupting the transcription and translation machinery. For example, the methods can include performing cycles of sonication for a total time ranging between two minutes and thirty minutes, or for up to one hour, and in some embodiments the total time period can be 1, 2, 3, 4, 5, or 6 hours. For example, the methods can include performing cycles of sonication for twenty minutes. In some implementations, the methods can include performing cycles of sonication for greater than thirty minutes. In some implementations, the methods can include performing cycles of sonication in time intervals. In a non-limiting example, the methods can include pulsing sonication for ten seconds at a time and then stopping for a gap period or reducing the level of sonication to 0 to 50%, e.g., 10, 20, 30, 40, or 50%.

The methods can include performing sonication for ten seconds, and then a gap period during which time no sonication is applied for twenty seconds. In some implementations, the methods can include performing sonication for a period of time ranging between seven seconds and twelve seconds, and then a gap of performing no sonication for a period of time ranging between ten seconds and twenty seconds (for one complete on/off cycle). In some implementations, the methods include performing sonication for a number of cycles in a range of 10-1000 cycles, e.g., 10 cycles, 50 cycles, 100 cycles, 125 cycles, 150 cycles, 300 cycles, 400 cycles, 500 cycles, 600 cycles, 700 cycles, 800 cycles, 900 cycles, or 1000 cycles, wherein each cycle includes a plurality of predetermined time periods of sonication, which can all be the same and/or different lengths of time, separated by gaps of no sonication, e.g., for about 5, 10, 15, 20, 25, or 30 seconds, wherein the gaps can all be the same length of time or different lengths of time. Performing sonication in time intervals with certain predetermined gap periods, e.g., of can reduce the chances of overheating the samples.

The liquid-filled reservoir can maintain a variety of temperature ranges to produce extracts efficiently without disrupting the transcription and translation machinery. For example, the methods can include chilling the liquid-filled reservoir, within which the sample plate is held during sonication, below a temperature threshold adequate to avoid overheating the extracts as they are being prepared. For example, the liquid-filled reservoir can be chilled to a temperature, e.g., below 50° C., below 40° C., below 30° C., below 20° C., or below 10° C.

The methods described herein can produce extracts from cell suspensions having various volumes. For example, the methods described herein provide increased throughput and small sample volumes, e.g., for experimental purposes. In some implementations, the sample volumes can be large. As a non-limiting example, the sample volumes of cellular suspensions can be in a range of about 20 microliters and about 50 milliliters.

In some implementations, the cell extracts can be combined with other reagents to carry out separate cell-free protein synthesis reactions. For example, the cell extracts can be combined with other reagents after sonication.

### Systems for High-Throughput Cell Lysis

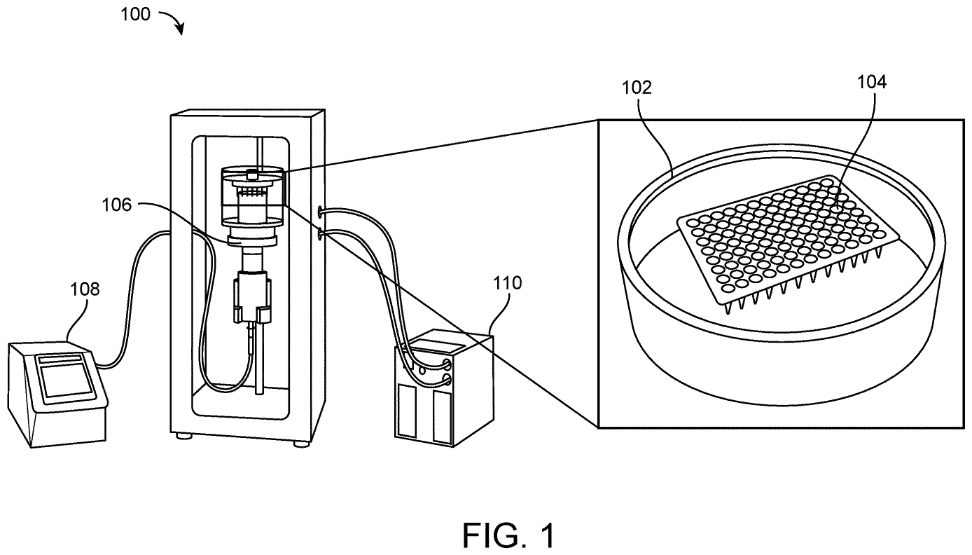

FIG. 1 is a schematic diagram of a system 100 that can be used to perform indirect sonication and cell lysis as described herein. The system 100 includes a reservoir 102 that can be filled with a liquid, such as water, and is arranged to hold a sample plate 104. The sample plate 104 can be, e.g., a 96-well plate, or a plate that holds a number of microtubes, etc. For example, the sample plate can include a sample plate that is standardized by the Society for Biomolecular Screening (SBS). Different types of sample plates may require different cell lysis parameters, as described further below. A sonic horn 106, e.g., an ultrasonic horn, is situated within or adjacent to the liquid-filled reservoir 102 so that the sonic horn 106 can apply sonic energy to the liquid in the reservoir 102. The sonic horn 106 can be, e.g., a sonotrode, that indirectly applies sonication to the sample plate 104 via the liquid in the reservoir 102.

Applying indirect sonication to the sample plate can enable a touchless lysis system, which can prevent loss of sample and cross-contamination. Indirect sonication can also enable a large number of samples, e.g., a 96-well plate, to be run in parallel, which increases throughput. For at least these reasons, indirect sonication is advantageous compared to direct sonication, which requires direct mechanical contact of a sonic transducer with each sample.

The sonic horn 106 is operatively connected to a power supply 108. In the illustrated example, the power supply 108 also acts as a signal generator. In some implementations, the system 100 includes a separate signal generator. The power supply 108 provides electrical signals to the sonic horn 106, which can receive the electrical signals from the power supply 108, convert the electrical signals into vibrational motion (e.g., via a transducer), and amplify the vibrations to apply the vibrations to the liquid in the reservoir 102.

A chiller 110 is controlled to actively cool the liquid in the liquid-filled reservoir 102 to counteract heat generated by sonication, e.g., as described above.

In some implementations, the system can include a sonicator system, such as the QSonica Q700MPX sonicator system, for example. However, the methods described herein are different from the manufacturer's cell lysis protocols. For example, the manufacturer's cell lysis protocols use a lower amplitude, a lower sonication time, different time intervals for sonication, and a different temperature than the methods described herein. The manufacturer's protocols also call for a bacterial suspension in 8M urea 4% SDS, which is a lysis buffer to aid in membrane disruption. This lysis buffer would be insufficient to generate cell extracts for use in cell-free protein synthesis because the lysis buffer may deactivate desired catalysts in the cell extract.

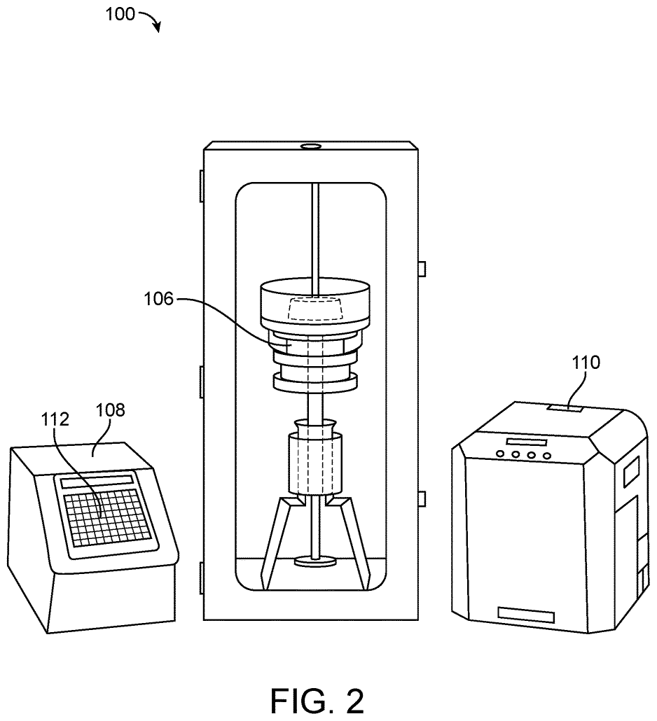

FIG. 2 is another schematic diagram of the system 100 of FIG. 1. Some portions of the system 100 are shown in greater detail in FIG. 2. For example, the power supply 108 includes a control panel 112 that allows a user to control the parameters of the sonication. In some implementations, the system includes a sound enclosure, e.g., to reduce sounds produced by the system 100.

The system 100 can include a number of different sonic horns. For example, the system can include a sonicator, such as the Q700 sonicator or Covaris R230. In other implementations, the system can include larger sonic horns, more powerful sonic horns, more or fewer sonic horns, etc. for different purposes. The type of sonic horn can change depending on a number of parameters, e.g., the number of samples being sonicated, the size of the samples being sonicated, etc. The methods described herein provide increased throughput and small sample volumes, e.g., for experimental purposes. However, modifications can be made to the methods to provide larger sample volumes.

A number of different types of liquid can be used in the liquid-filled reservoir. For example, the liquid can be water, e.g., sterile water, reverse osmosis water, distilled water, etc., or can be a buffer or cell growth medium. In other implementations, the liquid can include other solutions that transmit sonic energy, e.g., saline, gels, e.g., hydrogels, or other liquids, e.g., ethanol, plant oils, mineral oils, and organic solvents such as benzene, carbon tetrachloride, acetone, etc.

The system 100 can include different types of vessels to hold a different number of samples, different sizes of samples, etc. For example, the sample plate 104 can include a sample tray of 6, 24, 48, 96, 384, or 1536 wells. In some implementations, each well can have a volume that is less than 2 mL. In some implementations, the sample plate 104 can include eight-strip microtubes. In some implementations, the sample plate 104 can include more or fewer microtubes. The type, size, and numbers of vessels can change the parameters of the sonication. For example, different materials (e.g., different plastics) in the different vessels may respond to the sonication differently. Additionally, vessels having different wall thicknesses may respond to sonication differently. Vessels holding different sample sizes may also respond to sonication differently. Different numbers of vessels can also require more or less sonic energy during the sonication. The parameters of the methods for cell lysis can be modified to accommodate the changes in vessels. In some implementations, other components of the system 100 (e.g., the sonic horn 106) can be modified to accommodate the changes in vessels.

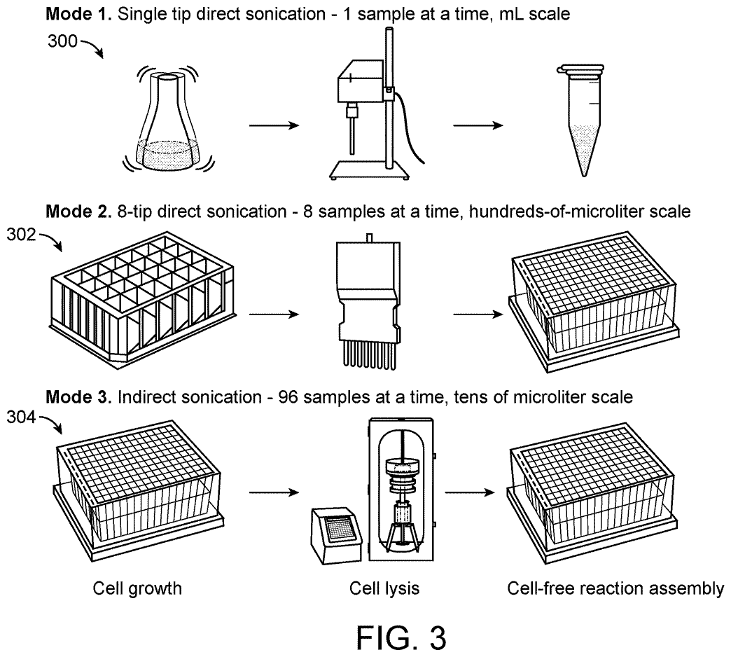

FIG. 3 illustrates the efficiencies and throughputs of different modes of direct sonication and a mode of indirect sonication. For example, a single-tip direct sonication method using a single tip sonication probe is shown at 300. The single-tip direct sonication can sonicate only one sample at a time because the single tip can be in direct contact with only a single sample. In addition, the single tip direct sonication can be used with larger sample volumes, e.g., around one milliliter. However, while useful for larger volumes, this mode of sonication is slow and not efficient for small volumes used for experimentation, because it generates only one extract at a time in larger volumes, which slows down the experimentation process. For example, cells in larger samples cannot be grown in parallel and require more processing for experimentation.

An eight-tip direct sonication method using an eight-tip sonication probe is represented in FIG. 3 at 302. The eight-tip direct sonication method can sonicate eight samples at a time because each tip of the eight-tip probe is in direct contact with a separate sample. The eight-tip direct sonication method can be used with samples that are hundreds of microliters in scale. This mode of sonication is advantageous over the single-tip direct sonication method 300, because it can produce more extracts, i.e., eight extracts, at a time, and this mode is slightly more efficient for experimentation because it creates more smaller-volume extracts than single-tip direct sonication. Of course, this method of direct sonication can be extended to probes with more tips, one for each sample, e.g., 12, 16, 20, 24, etc.

An indirect sonication method, as described herein, is represented in FIG. 3 at 304. In this example shown in FIG. 3, the indirect sonication method is illustrated using a 96-well sample tray. The indirect sonication method 304 can be used to sonicate 96 samples at a time, because a sonic horn is used to sonicate all of the individual samples simultaneously and indirectly via a liquid-filled reservoir. In addition, the indirect sonication can use samples that are tens of microliters in scale. Indirect sonication is advantageous over both single-tip direct sonication 300 and eight-tip direct sonication 302, because it can produce more extracts at a time. Indirect sonication is also advantageous over both single-tip direct sonication 300 and eight-tip direct sonication 302, because it can be used with much smaller sample volumes. Indirect sonication is further advantageous, because it avoids direct contact with the samples, which can prevent loss of sample and cross-contamination. Numerous other advantages have been made clear throughout the disclosure. This indirect method of sonication can also be easily reduced or expanded to sample trays with fewer or more sample wells, e.g., 6, 24, 48, 96, 384, or 1536 wells.

### EXAMPLES

### Example 1—Comparing Indirect Sonication to Direct Sonication

An experiment was conducted to compare the results of indirect sonication to the results of existing methodologies of direct sonication to determine whether indirect sonication is capable of generating active cell extracts for use in cell-free protein synthesis. The experiment was also designed to determine indirect sonication methods generate extracts with sufficiently low variability, e.g., for screening purposes. The indirect sonication extracts in the experiment were produced from a single indirect sonication run using the system and the methods described above. For example, the indirect sonication data was generated using the average activity of 96 extracts generated simultaneously using the QSonica Q700MPX sonication system.

The cells were suspended in acetate salt buffer, distributed equally across a 96-well plate, and placed in the sonication system. The 96-well plate contained 100 microliters of cellular suspension in each well. The sonication amplitude was 100% of the amplitude of the QSonica Q700 sonicator, which corresponds to a voltage output of 1000 VRMS. The observed power of the horn was approximately 620 W. The sonication process time, i.e., the total active time of the sonicator, was 20 minutes, with the sonic energy applied for ten seconds, and the sonic energy was off for gaps of twenty seconds.

The direct sonication extracts were produced via a single direct sonication process using an eight-tip probe. For example, the direct sonication data was generated using the average activity of eight different extracts generated simultaneously using an eight-tip direct sonication system. Each of the samples contained 100 microliters of cellular suspension. The sonication amplitude was 100% of the amplitude of the QSonica Q125 sonicator. The sonication protocol included applying sonic energy for a fifteen-second on period followed by a fifteen-second off period for a total of three on/off cycles. All of the extracts generated in this experiment were prepared from the same cellular material, distributed equally across the vessels. Because the indirect sonication extracts and the direct sonication extracts were generated and otherwise processed in the same fashion, the resulting difference in activity is primarily a function of the lysis method.

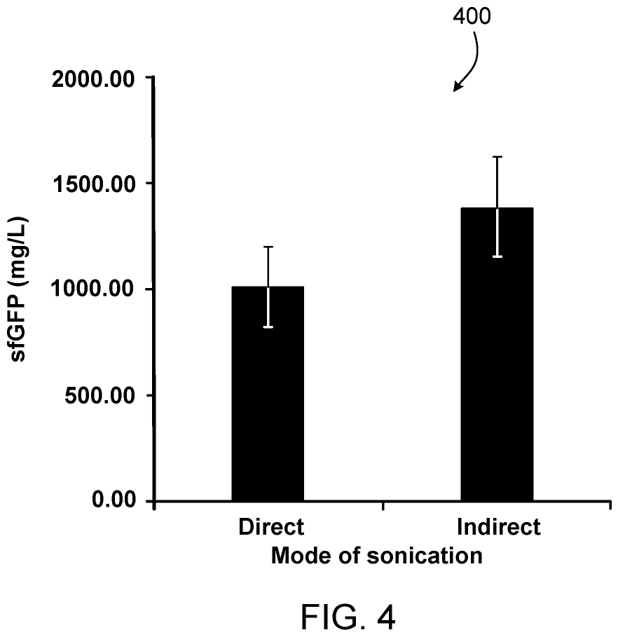

FIG. 4 is a plot 400 illustrating the average cell-free superfolder green fluorescent protein (sfGFP) reporter production enabled by the direct sonication extracts and the indirect sonication extracts prepared in the experiment. Other nucleic acid templates that encode for proteins can also be used. The error bars represent standard deviation. As illustrated, the sfGFP activity of the extracts prepared via indirect sonication is higher than the sfGFP activity of the extracts prepared via direct sonication. This illustrates that the methods described herein are capable of generating active cell extracts for use in cell-free protein synthesis.

Table 1 below compares the coefficients of variation resulting from indirect sonication and direct sonication. The coefficient of variation (CV=(std. deviation/average)*100)) measures the standard deviation of the activity of the extracts as a percentage of the mean.

TABLE 1

| Mode of sonication | Coefficient of variation |
|---|---|
| Direct | 18.1% |
| Indirect | 17.1% |

Table 1 illustrates that the methods described herein generate extracts with sufficiently low variability, e.g., for screening purposes, and is similar to the variability of direct sonication. The coefficients of variation result from sfGFP reporter production from cell-free protein synthesis reactions using extracts generated by the indicated mode of sonication (e.g., indirect sonication or direct sonication) across the throughput enabled by that mode of sonication. For example, indirect sonication as described herein enables sonication for a 96-well plate simultaneously. Direct sonication enables sonication for an eight-tip probe simultaneously. The coefficient of variation for direct sonication reflects the variability in sfGFP production between eight different extracts generated from the same cellular material using an eight-tip sonication probe. The coefficient of variation of indirect sonication reflects the variability in sfGFP production between 96 different extracts generated from the same cellular material, e.g., using the systems and methods described herein.

### Example 2—Comparing High-Throughput Indirect Sonication to High-Throughput Direct Sonication

An additional analysis was performed to compare equal numbers of extracts prepared via indirect sonication and prepared via direct sonication as an assessment of whether indirect sonication is capable of generating active cell extracts for use in cell-free protein synthesis at a similar level to direct sonication. The analysis also sought to compare the variability of indirect sonication to the variability of direct sonication at equal throughput. The indirect sonication extracts were produced simultaneously using the QSonica Q700MPX sonication system. The sonication amplitude was 100% of the amplitude of the QSonica Q700 horn, which corresponds to a voltage output of 1000 VRMS. The observed power of the horn was approximately 620 W. The sonication process time, i.e., the total active time of the sonicator, was 20 minutes, with the sonic energy applied for ten seconds, and the sonic energy was off for gaps of twenty seconds. The 96-well plate contained 100 microliters of cellular suspension in each well.

The direct sonication extracts were produced via twelve processes of an eight-tip sonication probe across a 96-well plate. The 96-well plate contained 100 microliters of cellular suspension in each well. The sonication amplitude was 100% of the amplitude of the QSonica Q125 sonicator, e.g., 380 VRMS at about 20 kHz.

The sonication protocol included applying sonic energy for a fifteen-second on period followed by a fifteen-second off period for a total of three on/off cycles. All of the extracts generated in this experiment were prepared from the same cellular material, distributed equally across the vessels. Because the extracts were generated and otherwise processed in the same fashion, the resulting difference in activity is primarily a function of the lysis method.

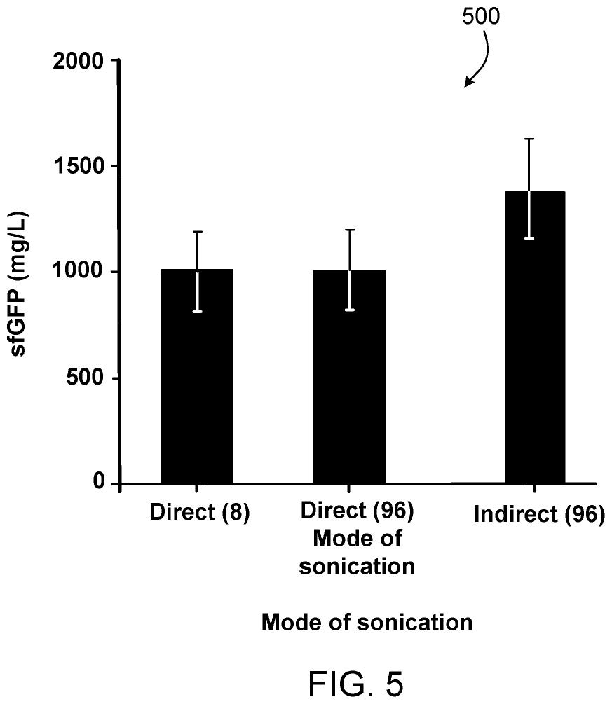

FIG. 5 is a plot 500 illustrating the average cell-free sfGFP reporter production produced by the direct sonication extracts and the indirect sonication extracts in the second analysis. The sfGFP production from the 96 individual extracts (e.g., a single process of a sonication system as described herein) was averaged. The average value of the indirect sonication described herein was compared to those of an eight-tip direct sonication at a throughput of eight extracts (e.g., a single process of an eight-tip sonication probe) and at a throughput of 96 extracts (e.g., twelve processes of an eight-tip sonication probe across a 96-well plate).

As illustrated, the sfGFP activity of the extracts prepared via indirect sonication is higher than the sfGFP activity of the extracts prepared via direct sonication at a throughput of eight extracts and the sfGFP activity of the extracts prepared via direct sonication at a throughput of 96 extracts. This illustrates that the methods described herein are capable of generating active cell extracts for use in cell-free protein synthesis. Additionally, the methods described herein increase the throughput of the cell lysis, e.g., because 96 samples can be prepared simultaneously rather than through twelve processes of an eight-tip sonication probe.

Table 2 below compares the coefficients of variation resulting from indirect sonication, direct sonication at a throughput of eight, and direct sonication at a throughput of 96.

TABLE 2

| Mode of sonication | Coefficient of variation |
|---|---|
| Direct (8) | 18.1% |
| Direct (96) | 16.9% |
| Indirect (96) | 17.1% |

Table 2 illustrates that the methods described herein generate extracts with sufficiently low variability, e.g., for screening purposes. The variability of indirect sonication is similar to the variability of direct sonication at a throughput of eight extracts and direct sonication at a throughput of 96 extracts. For example, the methods described herein have variability that is sufficiently low such that differences between extracts can reasonably be attributed to the extracts themselves, rather than attributed to the sonication process itself. The coefficients of variation result from sfGFP reporter production from cell-free protein synthesis reactions using extracts generated by the indicated mode of sonication (e.g., indirect sonication at a throughput of 96 extracts, direct sonication at a throughput of eight extracts, or direct sonication at a throughput of 96 extracts).

### OTHER EMBODIMENTS

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the implementations described above should not be understood as requiring such separation in all implementations.

Thus, particular implementations of the subject matter have been described. Other implementations are within the scope of the following claims. In some cases, the steps recited in the claims can be performed in a different order and still achieve desirable results. In addition, the processes depicted in the accompanying figures do not necessarily require the particular order shown, or sequential order, to achieve desirable results.

What is claimed is:

1. A method of lysing cells, the method comprising:
placing one or more vessels within a liquid in a reservoir, wherein each vessel contains one or more cells in a cell suspension; and
applying sonic energy to the liquid within the reservoir during a plurality of predetermined time intervals and under conditions sufficient to disrupt cell walls and/or cell membranes of cells in the cell suspension, and to release into the cell suspension cell contents that include active transcription and/or translation machinery.

2. The method of claim 1, wherein the sonic energy is applied indirectly to the cell suspension under conditions sufficient to maintain the functionality of the transcription and translation machinery contained within the cells.

3. The method of claim 1 or claim 2, wherein the sonic energy is applied indirectly to the cell suspension for a total time period of about 30 seconds to about 6 hours, e.g., about 30, 40, 50, 60, 70, 80, 90, 100, or 120 seconds, or about 1, 2, 3, 4, 5, 6, 7, 8, 9, 10, 15, 20, 25, 30, 40, 50, or 60 minutes, or about 1, 2, 3, 4, 5, or 6 hours.

4. The method of claim 3, wherein the predetermined time intervals are each about 5 to about 120 seconds, e.g., about 5, 10, 15, 20, 25, 30, 35, 40, 45, 50, 55, 60, 70, 80, 90, 100, 110, or 120 seconds, separated by gaps of about 5 to about 30 seconds, e.g., about 5, 10, 15, 20, 25, or 30 seconds, during which the sonic energy is not applied or is reduced.

5. The method of claim 4, wherein the predetermined time intervals are each about 5 seconds to about 20 seconds and the gaps between predetermined time intervals are about 10 to about 20 seconds, during which the sonic energy is not applied.

6. The method of any one of claims 1 to 5, wherein the total time period during which the sonic energy is applied is greater than or equal to 10 minutes and less than or equal to 20, 30, 40, 50 or 60 minutes.

7. The method of any one of claims 1 to 6, wherein the sonic energy is equal to an energy delivered by a sonicator with an amplitude set at about 50%.

8. The method of any one of claims 1 to 7, wherein a power density (W/mL reservoir) of the sonic energy is equal to the power density delivered by a sonicator with an amplitude set at about 50%.

9. The method of any one of claims 1 to 8, wherein an energy density (J/mL reservoir) of the sonic energy is equal to an energy density delivered by a sonicator with an amplitude set at about 50%.

10. The method of any one of claims 1 to 9, wherein a power density of the sonic energy is within a range of about 2 to about 3 W/mL of a volume of the reservoir.

11. The method of any one of claims 1 to 10, wherein an energy density of the sonic energy is within a range of about 2000 to about 5000 J/mL of a volume of the reservoir.

12. The method of any one of claims 1 to 11, further comprising maintaining a temperature of the liquid within the reservoir below a threshold temperature.

13. The method of claim 12, wherein the threshold temperature is at or below about 50° C., 40° C., 35° C., 30° C., 25° C., 20° C., 15° C., 10° C., or 5° C.

14. The method of claim 12 or claim 13, wherein the temperature of the liquid within the reservoir is maintained with a cooling system.

15. The method of any one of claims 12 to 14, wherein the temperature of the liquid within the reservoir is maintained by controlling a length of the predetermined time intervals and/or a length of the gaps between the predetermined time intervals.

16. The method of any one of claims 1 to 15, further comprising transmitting the sonic energy with sonic horn.

17. The method of any one of claims 1 to 16, wherein the vessels are part of a sample tray or plate.

18. The method of claim 17, wherein the sample tray or plate comprises a multi-well plate.

19. The method of claim 18, wherein the multi-well plate comprises 6, 24, 48, 96, 384, or 1536 wells.

20. The method of any one of claims 1 to 19, wherein the cells comprise bacterial cells, yeast cells, mammalian cells, insect cells, plant cells, and/or worm cells.

21. The method of any one of claims 1 to 20, wherein a frequency of the sonic energy is in a range of about 15 and about 30 kHz.

22. The method of any one of claims 1 to 21, wherein volumes of the cell suspensions are in a range between about 20 microliters and about 50 milliliters.

23. The method of any one of claims 1 to 22, wherein the liquid within the reservoir comprises one or more of water, one or more gels, and/or one or more organic solvents.

24. The method of any one of claims 1 to 23, wherein the vessels each have a volume of less than 2 milliliters.

25. A method of cell-free protein synthesis, the method comprising:
placing one or more vessels within a liquid in a reservoir, wherein each vessel contains one or more cells in a cell suspension;
applying sonic energy to the liquid within the reservoir during a plurality of predetermined time intervals and under conditions sufficient to disrupt cell walls and/or cell membranes of cells in the cell suspension, and to release into the cell suspension cell contents that include active transcription and/or translation machinery;
harvesting and/or processing at least a portion of the cell contents; and
synthesizing proteins using the active transcription and/or translation machinery.

26. The method of claim 25, wherein the active transcription and/or translation machinery comprises one or more of ribosomes, tRNAs, synthetases, transcription factors, translation initiation factors, elongation factors, or release factors.

27. The method of claim 25, wherein the active transcription and/or translation machinery comprises ribosomes, tRNAs, synthetases, transcription factors, translation initiation factors, elongation factors, and release factors.

28. The method of any one of claims 25 to 27, further comprising maintaining a temperature of the liquid within the reservoir below a threshold temperature at or below about 50° C., 40° C., 35° C., 30° C., 25° C., 20° C., 15° C., 10° C., or 5° C.

29. The method of any one of claims 25 to 28, wherein synthesizing the proteins comprises combining the portion of the cell contents with one or more nucleic acid templates that encode one or more proteins.

30. The method of any one of claims 25 to 29, wherein the sonic energy is applied indirectly to the cell suspension under conditions sufficient to maintain the functionality of the transcription and translation machinery contained within the cells.

31. The method of any one of claims 25 to 30, wherein the sonic energy is applied indirectly to the cell suspension for a total time period of about 30 seconds to about 6 hours, e.g., about 30, 40, 50, 60, 70, 80, 90, 100, or 120 seconds, or about 1, 2, 3, 4, 5, 6, 7, 8, 9, 10, 15, 20, 25, 30, 40, 50, or 60 minutes, or about 1, 2, 3, 4, 5, or 6 hours.

32. The method of claim 31, wherein the predetermined time intervals are each about 5 to about 120 seconds, e.g., about 5, 10, 15, 20, 25, 30, 35, 40, 45, 50, 55, 60, 70, 80, 90, 100, 110, or 120 seconds, separated by gaps of about 5 to about 30 seconds, e.g., about 5, 10, 15, 20, 25, or 30 seconds, during which the sonic energy is not applied or is reduced.

33. The method of claim 32, wherein the predetermined time intervals are each about 5 seconds to about 20 seconds and the gaps between predetermined time intervals are about 10 to about 20 seconds, during which the sonic energy is not applied.

34. The method of any one of claims 25 to 33, wherein the total time period during which the sonic energy is applied is greater than or equal to 10 minutes and less than or equal to 20, 30, 40, 50 or 60 minutes.

35. The method of any one of claims 25 to 34, wherein the sonic energy is equal to an energy delivered by a sonicator with an amplitude set at about 50%.

36. The method of any one of claims 25 to 35, wherein a power density (W/mL reservoir) of the sonic energy is equal to the power density delivered by a sonicator with an amplitude set at about 50%.

37. The method of any one of claims 25 to 36, wherein an energy density (J/mL reservoir) of the sonic energy is equal to an energy density delivered by a sonicator with an amplitude set at about 50%.

38. The method of any one of claims 25 to 38, wherein a power density of the sonic energy is within a range of about 2 to about 3 W/mL of a volume of the reservoir.

39. The method of any one of claims 25 to 38, wherein an energy density of the sonic energy is within a range of about 2000 to about 5000 J/mL of a volume of the reservoir.

40. The method of any one of claims 28 to 39, wherein the temperature of the liquid within the reservoir is maintained with a cooling system.

41. The method of any one of claims 28 to 39, wherein the temperature of the liquid within the reservoir is maintained by controlling a length of the predetermined time intervals and/or a length of the gaps between the predetermined time intervals.

42. The method of any one of claims 25 to 41, further comprising transmitting the sonic energy with sonic horn.

43. The method of any one of claims 25 to 42, wherein the vessels are part of a sample tray or plate, e.g., a multi-well plate.

44. The method of any one of claims 25 to 43, wherein the cells comprise bacterial cells, yeast cells, mammalian cells, insect cells, plant cells, and/or worm cells.

45. The method of any one of claims 25 to 44, wherein a frequency of the sonic energy is in a range of about 15 and about 30 kHz.

46. The method of any one of claims 25 to 45, wherein volumes of the cell suspensions are in a range between about 20 microliters and about 50 milliliters.

47. The method of any one of claims 25 to 46, wherein the liquid within the reservoir comprises one or more of water, one or more gels, and/or one or more organic solvents.

48. The method of any one of claims 25 to 47, wherein the vessels each have a volume of less than 2 milliliters.
